(19) United States
(12) Patent Application Publication
(10) Pub. No.: US 2017/0247741 A1
(43) Pub. Date: Aug. 31, 2017

(54) Oral Biofilm Models and Uses Thereof

(71) Applicant: Colgate-Palmolive Company, New York, NY (US)

(72) Inventors: Luciana Rinaudi Marron, Somerset, NJ (US); Richard SULLIVAN, Atlantic Highlands, NJ (US); David SURIANO, Edison, NJ (US); Aarti REGE, East Windsor, NJ (US); Ralph Peter SANTARPIA, Edison, NJ (US)

(73) Assignee: Colgate-Palmolive Company, New York, NY (US)

(21) Appl. No.: 15/512,125

(22) PCT Filed: Oct. 6, 2014
(86) PCT No.: PCT/US2014/059376

§ 371 Date: Mar. 17, 2017

(51) Int. Cl. C12Q 1/18; G01N 1/28; C12N 1/20

(52) U.S. Cl. CPC C12Q 1/18; C12N 1/20; G01N 1/2806

(57) ABSTRACT

The present disclosure provides an oral biofilm model including a substrate including a first surface, a second surface, and a plurality of specimens fixedly attached to the first surface, wherein an oral biofilm is capable of forming on the specimen. The surface roughness of at least one of the specimens of the plurality is less than or greater than a surface roughness of at least a second specimen of the plurality. The oral biofilm model also includes a body having sides and a bottom defining a vessel. The body is adapted to receive the substrate and the plurality of specimens and is further adapted to receive a fluid. Methods of forming oral biofilms and methods for identifying an agent for reducing or inhibiting biofilm formation are also provided.

### BACKGROUND

Biofilms are defined as sessile communities characterized by cells that are irreversibly attached to a surface or to each other, embedded in a matrix of extracellular polymeric substances. A biofilm community can be formed by a single kind of microorganism, but in nature, biofilms almost always consist of mixtures of many species of bacteria. For example, over 500 bacterial species have been identified in typical dental plaque biofilms.

The initiation and growth of dental plaque in vivo, as well as biofilms in general, consist of several phases and is a complex process dictated by many factors of both biological and physicochemical origin. Fluid dynamics also play a role in bacterial attachment and biofilm formation. In the early stages of biofilm formation in mammals, for example, surface topography may also be a major factor that dictates the adherence of bacteria to a surface. Rough surfaces may tend to accumulate more bacteria over a given time period than smooth surfaces due to the increased surface area of a rough surface.

However, in vitro oral biofilm models, which may be used to evaluate biofilm formation do not account for saliva flow and shear conditions. Accordingly, such models are more adequate for the study of periodontal pathogens rather than the study of plaque associated pathogens and plaque formation. Moreover, studies using conventional in vitro biofilm models have demonstrated no correlation between increased surface roughness and increased biofilm accumulation. Consequently, there remains a need for oral biofilm models that allow for a more realistic analysis of biofilm formation on teeth or implant surfaces and methods for evaluating polishing formulations.

### BRIEF SUMMARY

The present disclosure is directed to an oral biofilm model including: a substrate including a first surface, a second surface, and a plurality of specimens fixedly attached to the first surface, wherein an oral biofilm is capable of forming on the specimens, and wherein a surface roughness of at least one of the specimens of the plurality is less than or greater than a surface roughness of at least a second specimen of the plurality; and a body having sides and a bottom defining a vessel, the body adapted to receive the substrate and the plurality of specimens and further adapted to receive a fluid.

In another aspect, the present disclosure is directed to a method for growing oral biofilms, which method includes: providing at least a first and a second specimen on a substrate, wherein the first specimen includes a surface roughness less than or greater than a surface roughness of the second specimen, wherein an oral biofilm is capable of forming on the specimens; providing a vessel including a liquid growth medium, wherein the liquid growth medium includes microorganisms capable of oral biofilm production; agitating the liquid growth medium; suspending the substrate including the at least first and second specimens in the vessel; and incubating the at least first and second specimen with the liquid growth medium including the microorganisms, thereby forming a biofilm on the at least first and second specimens.

In yet another aspect, the present disclosure is directed to a method for identifying an agent for reducing or inhibiting biofilm. formation, which method includes: providing at least a first and a second specimen on a substrate, wherein the first specimen includes a surface roughness less than or greater than a surface roughness of the second specimen, wherein an oral biofilm is capable of forming on the specimens; providing a vessel including a liquid growth medium, wherein the liquid growth medium includes microorganisms capable of dental biofilm production; contacting the at least first specimen with a test agent; agitating the liquid growth medium; suspending the at least first specimen after contact with the test agent and the second. specimen in the vessel; incubating the at least first specimen after contact with the test agent and the second specimen with the liquid growth medium including the microorganisms; and comparing the amount of biofilm formed on the at least first and second specimen, wherein a reduced amount of biofilm formation on the at least first specimen in comparison to the amount of biofilm formation on the at least second specimen indicates that the test agent reduces or inhibits biofilm formation.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 depicts an embodiment of a vessel.

FIG. 2 depicts an embodiment of a vessel and substrates.

FIG. 3 depicts an embodiment of substrates embedded with specimens.

FIG. 4a depicts a stratification of a three cell study, enamel brushed with Whitening toothpaste vs rough enamel vs polished enamel. FIG. 4b depicts a stratification of a two cell study, enamel brushed with Sensitive toothpaste vs rough enamel.

FIG. 5 shows representative confocal images of enamel surfaces at 100× magnification. FIG. 5a: acid etched enamel, FIG. 5b: polished enamel, FIG. 5c: acid etched enamel brushed with the test. Whitening Toothpaste, FIG. 5d: acid etched enamel brushed with the test Sensitive Toothpaste.

FIG. 6 shows total bacterial accumulation normalized to surface area over a 6 hour period on enamel blocks.

FIG. 7 shows total bacterial accumulation normalized to surface area over a 6 hour period on enamel blocks

### DETAILED DESCRIPTION

The following description is merely exemplary in nature and is in no way intended to limit the disclosure, its application, or uses.

As used throughout, ranges are used as shorthand for describing each and every value that is within the range. Any value within the range can be selected as the terminus of the range. in addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

The Oral Biofilm Model

The present disclosure relates to a fixed volume, dynamic oral biofilm model, methods for assessing the formation of oral biofilms on specimens with varying surface roughness and methods for testing agents, such as oral product compositions, using the oral biofilm model.

As used herein, oral biofilms refer to three-dimensional structured bacterial communities which are embedded in an exo-polysaccharide matrix and attached to a solid surface, such as tooth enamel, the surface of a root or dental implants.

The oral biofilm model of the present disclosure includes specimens, which arc adhered to a substrate. As used herein, the term “specimen” refers to a natural or synthetic material on which an oral biofilm may be formed. Examples of natural specimens include extracted mammalian teeth, mammalian enamel and mammalian dentin. The natural specimens may be obtained from any mammal including but not limited to humans, non-human primates, camels, cats, chimpanzees, chinchillas, cows, dogs, goats, gorillas, horses, llamas, mice, pigs, murine, rats and sheep. Extracted mammalian teeth, such as bovine and/or human teeth are commercially available. Extracted human teeth may also be obtained from dental offices. In some embodiments, the natural specimens are bovine enamel.

Useful synthetic materials for specimens include those which are used to form dental implants, e.g., titanium, ceramics. Other synthetic materials which permit biofilm formation include but are not limited to synthetic hydroxyapatite, glass, silicon, urethane, or. similar materials.

The synthetic material specimens may be of any shape including in the form of geometric shapes, such as a square or a cylinder. The specimens may also include, for example, glass or plastic beads or discs, in some embodiments, the synthetic material is modeled to form a mammalian tooth.

The specimens are fixedly attached to a substrate using any means known in the art including the use of adhesives such as biocompatible adhesives, e.g., dental adhesives. In some embodiments, the specimens are adhered to a substrate using modeling clay. In other embodiment, silicone modeling puddy or other casting resins can be used.

In some embodiments, more than one specimen is adhered to a substrate, such as at least 2, 4, 6, 12, 24, 36, 50, 60 or more specimens. Accordingly, a substrate may contain a plurality of specimens affixed thereto.

In some embodiments, at least a surface roughness of at least a first one of a plurality of specimens is less than or greater than a surface roughness of at least a second one of the plurality of specimens. That is, all of the specimens may share the same surface roughness, while one of the specimens has a surface roughness, which is greater or less than the remainder of the plurality of specimens on a substrate. Alternatively, 1, 2, 3, 4, 5, 10, 20, 50, 100 or more of the specimens adhered to the substrate may share the same surface roughness, while the remainder of the plurality of specimens on a substrate share a different surface roughness. In other embodiments, each of the plurality of specimens has a different surface roughness. In yet other embodiments, a substrate will have specimens where there are at least 1, 2, 3, 4, 5, 6, 10, 20, 50, 80 or 100 or more surface roughness values which are different from the surface roughness values in the remainder of the plurality of specimens.

“Surface roughness” as used herein refers to the microscopic structural texture of a specimen surface. Surface roughness can be measured in terms of a number of parameters known in the art, including, but not limited to, average surface roughness, Ra; Rq (also called RMS; root mean square roughness); Rt (maximum roughness depths on the sample surface); Rz (average maximum peak to valley heights); and Rmax (maximum surface roughness). Surface roughness can be measured in terms of average surface roughness, Ra. Ra is the arithmetic average height of roughness component irregularities from the mean line measured within the sampling length. Smaller Ra values indicate smoother surfaces. Surface roughness can be measured by any method known in the art for measuring Ra, such as surface profilometry, surface scanning methods, confocal microscopy, atomic force microscopy, and scanning electron microscopy. Surface roughness can be measured before or after at least one treatment session and prior to any subsequent substantial exposure to other agents, for instance, remineralizing solutions (including saliva), or test agents.

In some embodiments, average Ra values range from about 2500 nm to about 5 nm, from 2000 nm to about 110 nm, from about 1000 nm to about 40 nm, from about 750 nm to about 40 nm, about 250 nm to about 20 nm, from about 200 nm to about 60 nm, about 50 nm, about 40 nm or about 30 nm. In other embodiments, the average Ra is greater than about 250 nm.

The surface roughness of the specimens may be imparted by acid etching. For example, the specimens may be immersed in a solution containing 37 wt % phosphoric acid for one minute, In other embodiments, the specimens may be immersed in a solution containing a mixture of 5% citric acid for 30 seconds.

In yet other embodiments, the surface roughness of the specimens may be reduced by brushing the specimen after acid etching with an agent, which decreases the surface roughness to a desired surface roughness. The agent may contain, for example, hydrated silica, hydrated alumina, calcium carbonate or dicalcium phosphates. In some embodiments, the agent is in the form of a toothpaste, gel, liquid or cream.

The substrate may be a glass substrate, a metal substrate, a polystyrene substrate, a polyethylene substrate, a vinyl acetate substrate, a polypropylene substrate, a polymethacrylate substrate, a polyacrylate substrate, a polyethylene substrate, a polyethylene oxide substrate, a polysilicate substrate, a polycarbonate substrate, a polytetrafluoroethylene substrate, a fluorocarbon substrate, a nylon substrate, a silicon substrate a rubber substrate, a polyanhydride substrate, a polyglycolic acid substrate, a polyhydroxyacid substrate, a polyester substrate, a polycapralactone substrate, a polyhydroxybutyrate, a polyphosphazene, a polyorthoester, a polyurethane, silicon casting resins or other casting resins, and combinations thereof.

In embodiments, substrates utilized in the oral biofilm model of the present disclosure have surface areas between 100 mm² and 3000 mm², typically between about 100 mm² and 2500 mm², more typically between 2200 mm² and 500 mm², and still more typically between 2000 mm² and 500 mm². In some embodiments, the substrate is in the size and shape of a microscope slide, e.g. a glass microscope slide, typically 25 mm by 75 mm.

After the specimens have been fixed to the substrate, the specimens are suspended within a vessel containing a liquid growth medium. The vessel of the present disclosure may be designed to accommodate, e.g., 1, 2, 3, 4, 5, 6, 7, 8, or more separate substrates. In some embodiments, each substrate of the oral biofilm model of the present disclosure includes a surface having the same area, whereas in other embodiments, at least one substrate in the oral biofilm model of the present disclosure includes a surface area that is different from that of another substrate in the oral biofilm model.

The vessel is adapted to receive the support substrate and the affixed specimens in a fluid tight communication, which is capable of retaining a liquid growth medium therein. Appropriate vessels include, for example, commercially available 4-, 6-, 8-, 12-, 24-, 96-, or 384-well plastic tissue plates or Petri dishes, e.g. a 100×15 mm square Petri dish. Useful materials for the vessels include, but are not limited to, glass, polystyrene, polypropylene, polycarbonate, copolymers (e.g., ethylene vinylacetate copolymers), and the like.

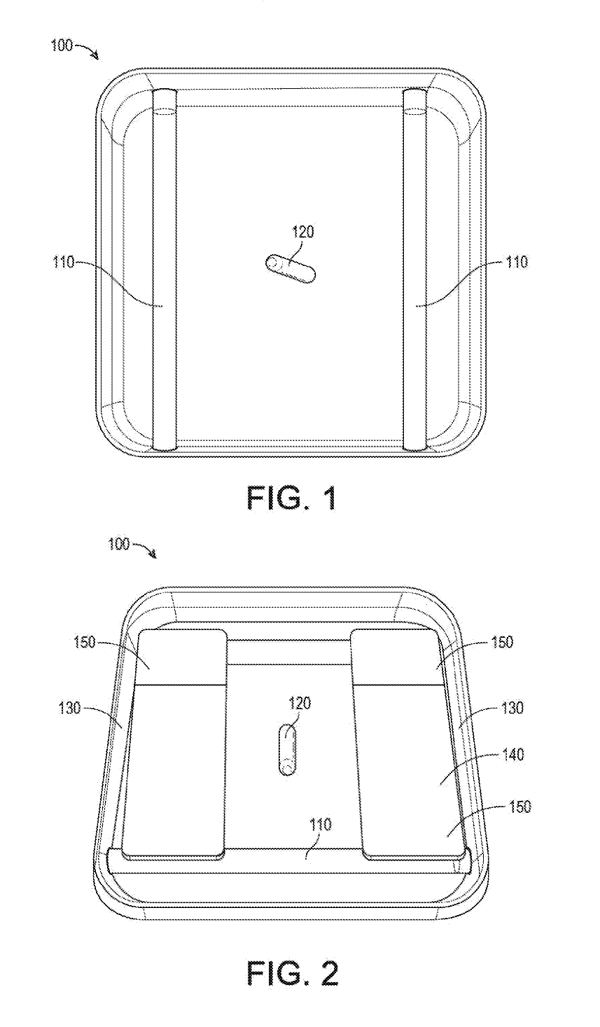

Referring now to FIG. 1, there is shown a view of a vessel (100) containing two substrate supports (110). The substrate supports are aligned in parallel along two sides of the vessel (100). In this embodiment, the substrate supports are 3.5 inch pipette pieces. However, any other suitable means to suspend the substrate and specimens may be used, e.g., the substrate supports may be integrally formed with the vessel (100) during the manufacturing process. In the embodiment shown in FIG. 1, a stirrer bar (120) is placed in the vessel to agitate the liquid growth medium causing the growth medium to move across the specimens.

Referring now to the FIG. 2, there is shown a view of a vessel (100) containing two substrates (130). The substrates contain a first surface (not shown) to which specimens are fixed and a second surface (140). Each distal end (150) of each substrate (130) is placed on each of the substrate supports (110). The first surface of the substrates to which the specimens are affixed are suspended in a liquid growth medium.

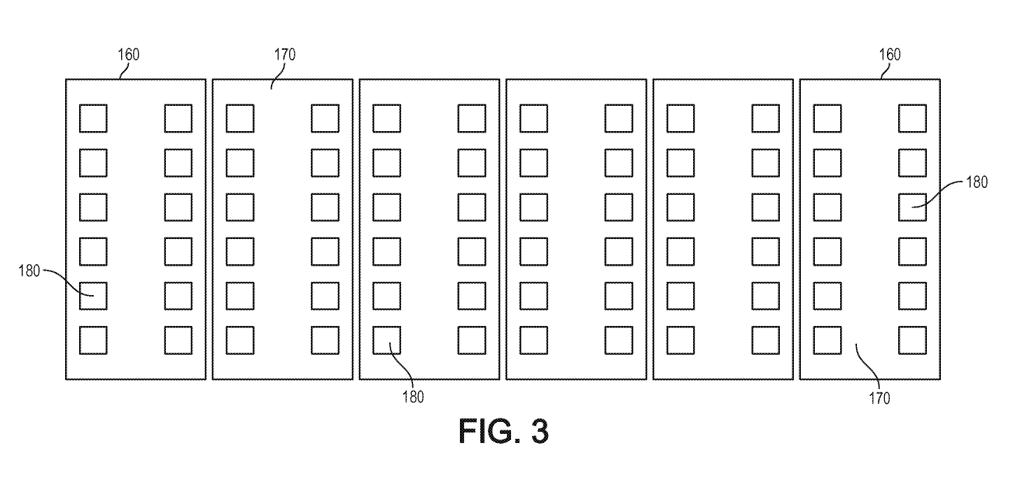

FIG. 3 depicts first surfaces (160) of the substrates with modeling clay (170) on the first surfaces (160). The specimens (180) are embedded in the modeling clay (170). In some embodiments, only one substrate is placed into the vessel. In other embodiments, two, three, four, ten or twenty substrates, each containing specimens (180) may be placed into a vessel.

The oral biofilm model described herein allows for specimens (180) having different surface roughness values to be simultaneously tested in a vessel (100) containing a liquid growth medium. The substrate (130) of the present disclosure allows the exposure time/growth. time of the biofilm to be carefully monitored and controlled by removing the entire substrate (130) from the vessel (100) wherein all of the specimens (180) are affixed to the substrate (130). Therefore, the process of removing the substrate (130) may correlate to removing all of the specimens (180) from a liquid growth media simultaneously. Thus, the substrate (130) promotes uniform. formation of biofilm on each of the specimens (180) because all of the specimens (180) may be removed from the vessel (100) in a single action. The production of uniform biofilms may ensure that test results are uniform and accurate. Still further, the oral biofilm model of the present disclosure allows for high throughput of biofilm formation because a large number of specimens (180) may be prepared at once.

The vessel (100), which serves as a reservoir for a liquid growth medium containing biofilm forming organisms, may generate a shear force across the specimens. The generated shear force allows for optimal biofilm formation on the specimens. The shear force developed in the vessel may be generated by a stirrer bar as shown in FIGS. 1 and 2 or may be generated by a rocking table or a gyrating shaker, for example. The vessel of the fixed volume, dynamic oral biofilm model described herein allows for a more realistic analysis of biofilm growth on specimens under flowing, aerobic conditions similar to what occurs in the mouth; while current static oral biofilms models known in the art do not account for saliva flow, shear, and oxygenation conditions.

Methods of Using Oral Biofilm Model

The fixed volume dynamic oral biofilm model of the present disclosure may be used to grow biofilms and assess the characteristics of the biofilms. For example, the effects of surface roughness on particular specimens, such as enamel specimens as described herein may be assessed. The specimens are incubated, for example at 37° C. under aerobic conditions in a vessel containing a liquid growth medium for a period of time to allow a biofilm to form on the specimen. The period of time allowed for biofilm formation ranges from about 2 hours to about 24 hours, about 3 hours to about 24 hours, about 3 hours to about 10 hours, about 4 hours to about 8 hours or may be about 6 hours. During incubation, biofilm formation may be promoted by providing agitation of the liquid growth medium, allowing the medium to flow across the specimens. For example, a stirrer bar, rocking table or a gyrating shaker as described above may be included in the vessel to promote agitation. After formation of a biofilm, the biofilm may be removed from the specimen by sonication for example, to assess, e.g., the amount of colony forming units (CFU).

The liquid growth medium, which may be used with the model and methods described herein may be any liquid growth medium known in the art for growing biofilms. For example, brain heart infusion medium (Sigma-Aldrich, St. Louis, Mo.) supplemented with human serum (Sigma-Aldrich), (4:1) saliva-like medium (SLM, 0.1% Lab Leraco Powder, 0.2% yeast extract, 0.5% peptone, 0.25% mucin from porcine stomach, type III (Sigma-Aldrich), 6 mM NaCl, 2.7 mM KCl, 3.5 mM KH₂PO₄, 1.5 mM K₂HPO₄, 0.05% urea, pH 6.7) (1:3) may be used. Alternatively, a chemically defined medium (CDM) may be used without any glucose or supplemented with either human serum (4:1), 50 mM glucose or 50 mM sucrose, see Rijn and Kessler, Infect Immun., 1984, 27(2):444-448 incorporated herein by reference. In some embodiments, McBain medium is used, supplemented with sucrose, heroin, vitamin K, and fresh or frozen saliva, see McBain et al., 2005, “Development and characterization of a simple perfused oral microcosm”, J. Appl. Microbiol, 98, 624-634, which is incorporated herein by reference. In some embodiments, the liquid growth medium comprises glucose or sucrose.

The oral biofilm model of the present disclosure is suitable for formation of biofilms caused by plaque-producing microorganisms and/or the formation of biofilms caused by microorganisms responsible for periodontal disease. In some embodiments, the model may be used for the formation of biofilms caused by plaque-producing microorganisms.

In some embodiments, the liquid growth medium contains one or more biofilm forming organisms. In some embodiments, the biofilm forming microorganisms are those belonging to the genera, which are associated with periodontal disease, which include but are not limited to the Treponema, Bacteroides, Porphyromonas, Prevotelia, Capnocytophaga, Peptostreptococcus, Fusobacterium, Actinobacillus, and Eikenella. In other embodiments, the liquid growth medium contains one or more periodontal associated species, such as Treponema denticola, Porphyromonas gingivalis, Bacteroides forsythus, Prevotella intermedia, Prevotella nigrescens, Peptostreptococcus micros, Fusobacterium nucleatum subspecies, Eubacterium nodatum or Streptococcus constellatus.

In other embodiments, the liquid growth medium contains at least one microorganism associated with dental plaque formation selected from the genera: Streptococcus, Veillonella, Actinomyces, Granulicatella, Leptotrichia, Lactobacillus, Thiomonas, Bifidobacterium, Propionibacterium or Atopobium. In other embodiments, the liquid growth medium contains one or more species associated with dental plaque formation including but not limited to Streptococcus mutans, Streptococcus sobrinus, Streptococcus gordonii, Streptococcus sanguinis, Lactobacillus acidophilus, Lactobacillus casei, Lactobacillus fermentum, Lactobacillus delbrueckii, Lactobacillus plantarum, Lactobacillus jensenii, Lactobacillus brevis, Lactobacillus salivarius, Lactobacillus gasseri and Actinomyces naeslundii. In other embodiments, the liquid growth medium at least contains Streptococcus mutans.

In some embodiments, the liquid growth medium may contain saliva from a mammalian donor, such as humans, non-human primates, camels, cats, chimpanzees, chinchillas, cows, dogs, goats, gorillas, horses, llamas, mice, pigs, murine, rats and sheep. In some embodiments, human saliva is used.

Using the oral biofilm model of the present disclosure, the effects of surface roughness, on biofilm formation may be assessed. Assessment of the biofilm formation may be determined by, for example, the use of confocal laser scanning microscopes to observe biofilm morphology and/or adherence to the specimen surface. The number of colony forming units in each of the formed biofilms may also be determined. Enumeration of bacteria present in the biofilms can also be achieved by using molecular approaches such as quantitative polymerase chain reaction (qPCR or Real-Time PCR).

In addition, the oral biofilm model of the present disclosure may be used to test the efficacy of test agents, such as oral care products, in the form of a toothpaste, a gel, a mouthwash, a powder or a cream, for example, on the surface roughness of the specimen to assess their effects on biofilm formation. For example, after acid-etching, a test agent, such as an oral care product, may be brushed onto the specimen. After brushing, the specimens may be suspended in the liquid growth medium and incubated. After incubation, the biofilms may be assessed by confocal scanning microscopy, by determining the number of colony-forming units (CFU), or by qPCR to determine the efficacy of the test agent for reducing or inhibiting oral biofilms.

The oral biofilm model of the present disclosure is not limited to use in testing variance of surface roughness on biofilm formation or testing agents in combination with specimens having various surface roughness values. The oral biofilm model of the present disclosure can readily be used to compare the effects on biofilm formation of, for example, different microorganisms, different specimens and/or different liquid growth media and/or test agents and/or varying surface roughness of the specimens. For instance, when the vessel of the oral biofilm model of the present disclosure is a multi-well plate, liquid growth media containing different microorganisms can be incorporated into each of the wells. The effects of the different microorganisms, alone, or in combination with surface roughness may then be assessed using the oral biofilm model described herein.

### EXAMPLES

### Example 1

### Preparation of Enamel Specimens

Precut bovine enamel specimens were Obtained from Bennet Amaechi, DDS, MS, PhD, FDI, Professor and Director of Cariology, Department of Comprehensive Dentistry, University of Texas Health Science Center at San Antonio, 7703 Floyd Curl Drive, MC 7917, San Antonio, Tex. 78229-3900. The specimens were cast into a 38 mm diameter disk using an acrylic casting resin to enable the specimens to be polished to a mirror finish using a Buehler polisher.

The specimens were visually inspected to ensure the enamel was fully exposed and free of defects. Each disk held approximately 18 to 20 specimens. The specimens were divided into three groups: polished, acid etched, acid etched plus brushing with the test toothpastes. Acid etching was accomplished by immersing the specimens in 5% citric acid for 30 seconds. A subset of the acid etched specimens was brushed on a Kal-Tech linear brushing machine using a 1:3 slurry of test toothpastes. An ordinary flat trimmed toothbrush was used to brush specimens. The brush tension adjusted to 280 g downward pressure and specimen were brushed for 4500 strokes.

The surface roughness of the individual specimens were measured using a Leica DCM3D confocal microscope using blue light and a 100× (NA 0.9) EP1-L lens. Ra values were obtained by analyzing the topographical images using Leica Map software. Surface area measurements of the individual enamel specimens were measured using an Olympus BX60 microscope operating in bright field mode. The specimens were viewed with an Olympus MPlanAPO 1.25×/0.04 lens. Images of the specimens were captured using a Hitachi KP-M1U CCD camera and a Scion Image PCI Frame grabber. Area calculations were made using Scion Image 4.0 software. A hydroxyapatite disk of known diameter (5 mm) was used to calibrate the area measurements and convert pixels into surface area units (mm²).

### Example 2

### Bacterial Attachment to Enamel Specimens as a Function of Surface Roughness (Polished, Acid Etched, and Acid Etched/Brushed with Test Toothpastes)

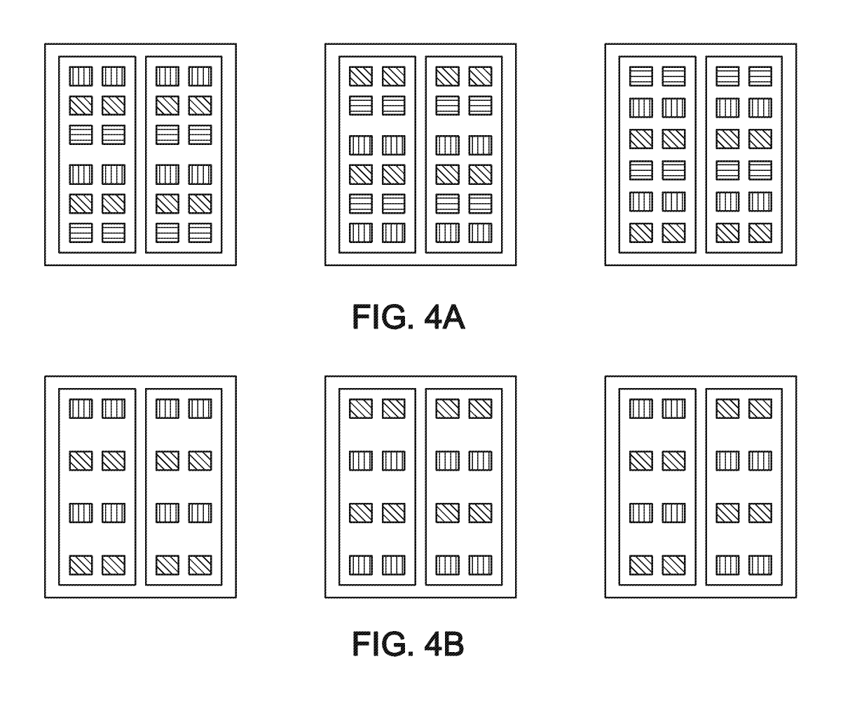

Bacterial attachment studies were conducted using a fixed volume dynamic flow oral biofilm model. In this model, the reaction vessel was a 100×15 mm square polystyrene Petri dish (Electron Microscopy Science). Enamel specimens were mounted on a microscope slide using modeling clay. Care was taken to ensure only the conditioned enamel surface was exposed and the clay surface with the enamel specimens was as flat as possible to minimize variations from specimen to specimen and slide to slide in turbulent flow. For a particular experimental run, three reaction vessels were used. Each vessel contained a maximum of 24 enamel specimens (two slides with 12 specimens each). Experiment 1, a three cell study was conducted: rough vs polished vs the Whitening Toothpaste. For Experiment 2, a two cell study was conducted: rough vs the Sensitive Toothpaste to simplify the experiment and increase statistical power. For a three cell study, it was determined that two runs (n=48) would be needed to achieve sufficient power to see significant differences among treatments. For the two cell study, a single run (N=24) was sufficient to see significant treatment differences. The distribution of the enamel specimens was stratified among the three reaction vessels (labeled Red, Green, and Blue for the three cell study and labeled Red, Green, and ted/Green for the two cell study) to account for positional effects associated with flow. The stratification is shown in FIG. 4a for the three-cell and FIG. 4b for the two cell study. Different colored blocks represent the different treatments among the enamel specimens.

### Example 3

### Protocol for Growing and Quantifying Polymicrobial Biofilms on Enamel Blocks

A. Semi-Dynamic System Preparation

A polystyrene pipette was cut into two 3.5 in pieces. The pipette pieces were made to fit tightly inside the square vessel. The pipette pieces were disinfected with a 1:10 bleach solution for 30 minutes, rinsed thoroughly with sterile water and then left to dry. The specimens were embedded (12/slide) on top of a substrate (microscope slide) containing an evenly distributed layer of modeling clay as shown in FIG. 3. The blocks were stratified so that specimens from different treatments were present in each slide to balance out any possible positional effect. The microscope slides were UV sterilized (enamel blocks side up) for 30 minutes. Under sterile conditions, the two pieces of pipette were placed inside a square dish (vessel) containing a small sterile stirrer bar as shown in FIG. 1. The microscope slide was transferred to the vessel so that it was seated upside down on top of the pipettes as shown in FIG. 2. The vessel was UV sterilized for another 30 minutes.

B. Saliva Collection

The teeth of the human donor for saliva collection were not brushed on the evening before and on the day of sampling. The sample was taken at least two hours after the last meal and/or drink. The saliva donor was asked to chew parafilm and saliva was collected in a sterile conical tube, which was kept on ice during saliva collection. Under sterile conditions, the saliva sample was diluted 1:1 with 60% sterile glycerol. The saliva sample was diluted into 800 μl aliquots into sterile microcentrifuge tubes. The saliva sample was labeled and stored in tubes at −20° C. until use.

C. Dish Inoculation, and Incubation Conditions

A 50 ml sterile conical tube was prepared with 40 ml McBain medium, 400 μl 20% sterile sucrose, 80 μl 0.05% hemin, 1.6 μl 0.5% Vitamin and 800 μl fresh or or 1.6 ml of frozen saliva in glycerol. A vessel (square Petri dish) containing the specimens embedded in modeling clay was inoculated with the saliva-McBain mix. The vessel was incubated aerobically at 37° C., with gentle stirring for 6 hours.

D. Harvesting of the Biofilms, and Quantification of Colony Forming Units (CFU)

Under sterile conditions, the slide containing the specimens was removed from the vessel and submerged in 45 ml PBS pH 7.4 to remove planktonic cells. Each specimen was gently removed from the slide with sterile tweezers; and the specimen was transferred to 1 ml PBS pH 7.4 in a pre-labeled 24-well plate. The suspensions were sonicated for 2 min (30 second pulses). The suspensions were serially diluted in PBS pH 7.4 and plated on Trypticase Soy Agar, with 5% Sheep Blood (TSA II) plates for determination of CFU. Typical dilutions for counting are 10⁰-10⁻³. The plates were incubated aerobically at 37° C. for 48 h. The colony forming units (CFU) were determined by colony counting and the results were reported as CFU/ml.

Results and Summary

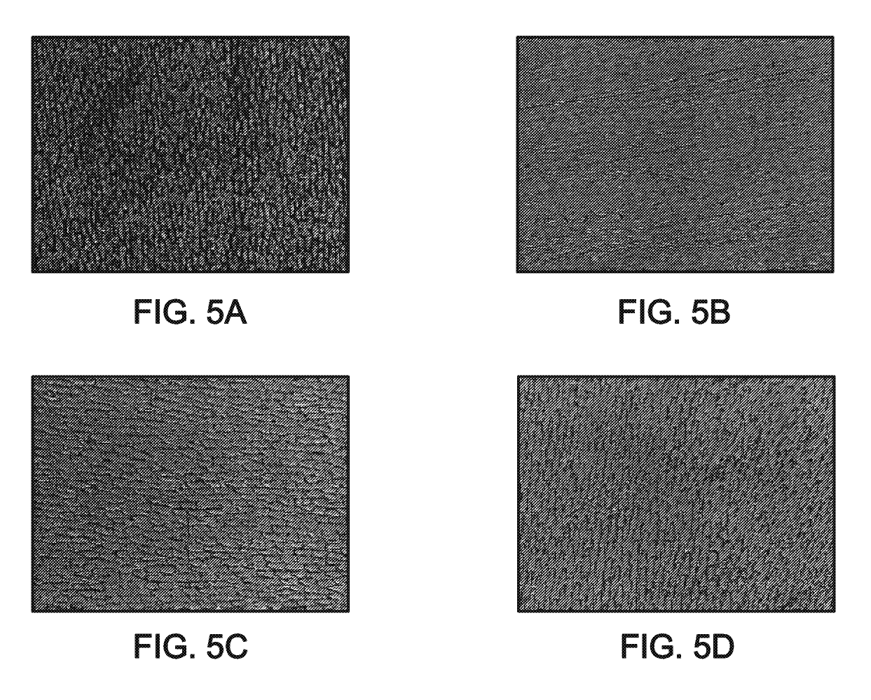

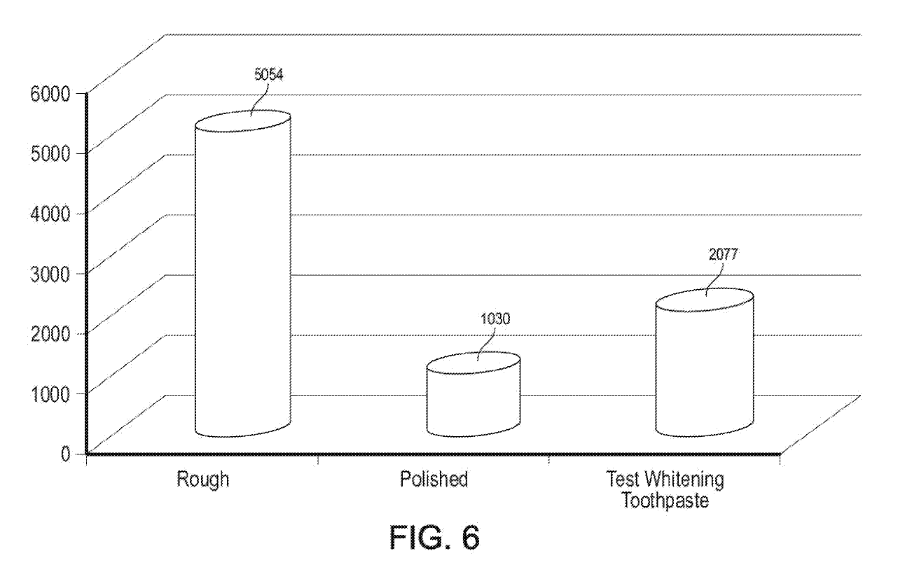

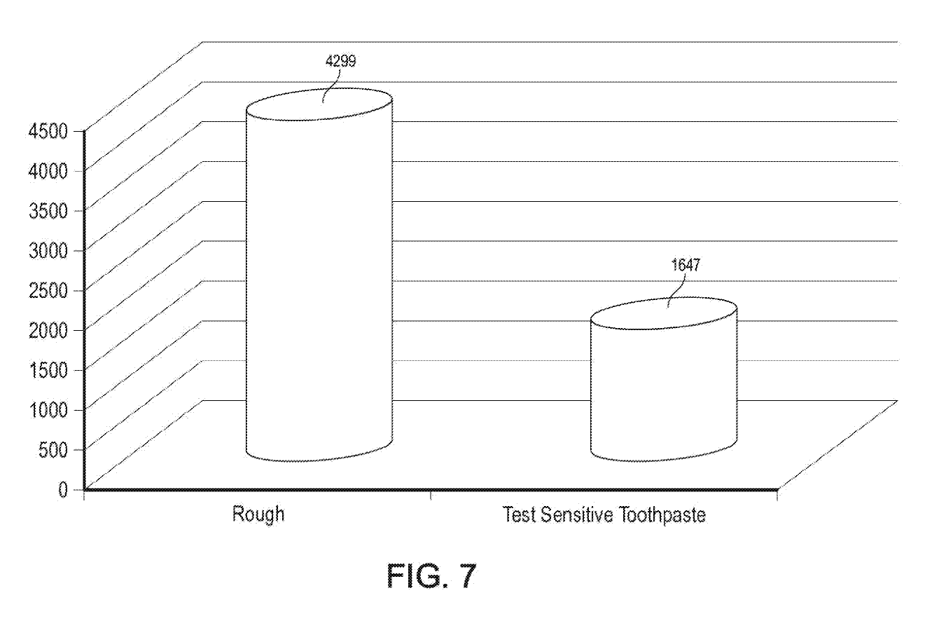

FIG. 5 shows representative confocal images of the polished enamel, acid etched enamel, and acid etched enamel after brushing with the test toothpastes. The Ra values for these representative images are 237 nm for rough, 26 nm for polished, 55 nm for Whitening Toothpaste, and 63 nm for Sensitive Toothpaste. From FIG. 5 and the Ra values, the acid etched surface clearly had the roughest surface topography, followed by the acid etched enamel brushed with test toothpastes, and then highly polished enamel surface. FIGS. 6 and 7 show the bacteria attachment results for Experiments 1 and 2. In Experiment 1, described above in Example 1, above, CFU values were successfully measured for 159 of the 164 enamel specimens and 43 of the 48 specimens tier Experiment 2. The inability to measure the CFU values was a result of bacterial contamination. In Experiment 1, the average bacteria counts normalized to surface area were 5054 CFU/mm² for rough, 1030 CFU/mm² for polished, and 2077 CFU/mm² for the Whitening Toothpaste. The ANOVA analysis showed that the treatment effect was statistically significant (p=0.001). Comparison among treatments using the Tukey Test showed that statistically significantly (p<0.05) more bacteria adhered over a 6 hour time period to the rough enamel in comparison to the Whitening Toothpaste and polished enamel surface. There was no statistically significant (p>0.05) difference between the Whitening Toothpaste treated etched enamel and the highly polished enamel surfaces. For Experiment 2, described in Example 1, above, rough enamel was compared to acid-etched enamel brushed with the Sensitive Toothpaste. Statistically significantly (p<0.05) more bacteria accumulate on the acid-etched surface compared to the Sensitive Toothpaste treated surface after 6 hours. The average bacterial accumulation normalized to the surface area was 4299 CFU/mm² for the acid-etched enamel and 1647 CFU/mm²for the Sensitive Toothpaste.

In summary, surface topography may be a key factor in bacterial adherence. In this study, the effect of enamel surface roughness on bacterial accumulation on the enamel surface over a 6 hour time period was explored. Three enamel surfaces were examined: highly polished (polished), acid etched (rough), and acid etched followed by brushing with either a toothpaste containing 0.243% NaF in a 10% HCS base (Whitening toothpaste) or 5% potassium nitrate, 0.243% Naf in a 10% HCS base (Sensitive toothpaste).

Analysis of the surface topographies of the three different enamel specimen treatment groups by confocal microscopy indicated clear differences in the roughness of acid-etched, polished, and brushed specimens. The acid etched enamel specimens clearly lost significant amounts of reflectivity due to roughening of the surface by the citric acid. Brushing the acid etched with either the Whitening or Sensitive toothpaste smoothed the enamel surface and helped restore the reflectivity of the surface. The polished enamel specimens were the smoothest and most reflective of the three surfaces, which is expected since this surface was polished using a very fine diamond paste. In Experiment 1, comparison of acid-etched, polished and Whitening Toothpaste brushed enamel showed that the quantity of bacteria found on the enamel surfaces was positively correlated with the roughness of the enamel: acid-etched enamel had the most bacteria, followed by brushed enamel, and then polished enamel. Acid-etched enamel had 4.9 times more bacteria per unit surface area compared to polished enamel and 2.4 times more bacteria per unit surface area compared to the Whitening Toothpaste. In Experiment 2, the acid-etched enamel had 2.6 times more bacteria per unit surface area compared to enamel brushed with Sensitive Toothpaste. The relative magnitudes of the differences between the rough and test toothpaste treatments were similar for Experiment 1 and 2. This is expected since the Whitening Toothpaste and Sensitive Toothpaste have an identical cleaning/polishing system and differ mainly in color and the presence of 5% KNO₃ in the Sensitive Toothpaste.

In summary, the results of this study confirm the positive relationship between surface roughness and bacterial adhesion on enamel surfaces. Exposure of enamel to acid roughens the surface and allows bacteria to accumulate more readily. The cleaning/polishing system used in the two test toothpastes is capable of polishing and smoothing enamel, resulting in the attachment of fewer bacteria compared to roughened acid-etched enamel.

What is claimed is:

1. An oral biofilm model comprising:
a substrate comprising a first surface, a second surface, and a plurality of specimens fixedly attached to the first surface, wherein an oral biofilm is capable of forming on the specimens, and wherein a surface roughness of at least one of the specimens of the plurality is less than or greater than a surface roughness of at least a second specimen of the plurality; and
a body having sides and a bottom defining a vessel, said body adapted to receive said substrate and said plurality of specimens and further adapted to receive a fluid.

2. The oral biofilm model of claim 1 wherein the substrate is a glass microscope slide.

3. The oral biofilm model of claim 1, wherein the specimens are synthetic specimens.

4. The oral biofilm model of claim 1, wherein the specimens are natural specimens.

5. The oral biofilm model of claim 4, wherein the natural specimens are selected from the group consisting of mammalian enamel, mammalian dentin and mammalian teeth.

6. The oral biofilm model of claim 5, wherein mammals of the mammalian specimens are selected from the group consisting of bovine, swine and human.

7. The oral biofilm model of claim 5, wherein the mammalian enamel is bovine enamel.

8. The oral biofilm model of claim 3, wherein the synthetic specimens are selected from the group consisting of synthetic hydroxyapaptite, glass and ceramic.

9. The oral biofilm model of claim 3, wherein the synthetic specimens are beads or discs.

10. The oral biofilm model of claim 1, wherein an average surface roughness (Ra) of the specimens ranges from 2500 nm to 5 nm.

11. The oral biofilm model of claim 1, wherein the surface roughness is formed by acid etching.

12. The oral biofilm model of claim 11, wherein the surface roughness is formed by acid etching followed by contact with an agent, which reduces the surface roughness of the specimen.

13. The oral biofilm model of claim 12, wherein the agent is a toothpaste.

14. A method of forming an oral biofilm, the method comprising:
providing at least a first and a second specimen on a substrate, wherein the first specimen comprises a surface roughness less than or greater than a surface roughness of the second specimen, wherein an oral biofilm is capable of forming on the specimens;
providing a vessel comprising a liquid growth medium, wherein the liquid growth medium comprises microorganisms capable of oral biofilm production;
agitating the liquid growth medium;
suspending the substrate comprising the at least first and second specimens in the vessel; and
incubating the at least first and second specimen with the liquid growth medium comprising the microorganisms, thereby forming a biofilm on the at least first and second specimens.

15. The method of claim 14, wherein the surface roughness is formed by acid etching.

16. The method of claim 14, wherein the incubating step is from about 3 hours to about 24 hours.

17. The method of claim 16, wherein the incubating step is about 6 hours.

18. The method of claim 14, wherein the specimens are natural specimens.

19. The method of claim 18, wherein the specimens are selected from the group consisting of at least one of mammalian enamel, mammalian dentin and mammalian teeth.

20. The method of claim 14, wherein the specimens are synthetic specimens.

21. The method of claim 14, wherein the average surface roughness (Ra) of the specimens ranges from 750 nm to 40 nm.

22. The method of claim 14, wherein the liquid growth medium comprises saliva.

23. The method of claim 14, wherein the microorganisms are selected from at least one of the group consisting of Streptococcus mutans, Streptococcus sobrinus, Streptococcus gordonii, Streptococcus sanguinis, Lactobacillus acidophilus, Lactobacillus casei, Lactobacillus fermentum, Lactobacillus delbrueckii, Lactobacillus plantarum, Lactobacillus jensenti, Lactobacillus brevis, Lactobacillus salivarius, Lactobacillus gasseri and Actinomyces naeslundii.

24. The method of claim 14, wherein the liquid growth medium comprises sucrose.

25. A method for identifying an agent for reducing or inhibiting biofilm formation, the method comprising:
providing at least a first and a second specimen on a substrate, wherein the first specimen comprises a surface roughness less than or greater than a surface roughness of the second specimen, wherein an oral biofilm is capable of for on the specimens;
providing a vessel comprising a liquid growth medium, wherein the liquid growth medium comprises microorganisms capable of oral biofilm production;
contacting the at least first specimen with a test agent;
agitating said liquid growth medium;
suspending said at least first specimen after contact with the test agent and the second specimen in the vessel;
incubating said at least first specimen after contact with the test agent and the second specimen with the liquid growth medium comprising the microorganisms; and
comparing the amount of biofilm. formed on the at least first and second specimen, wherein a reduced amount of biofilm formation on the at least first specimen in comparison to the amount of biofilm formation on the at least second specimen indicates that the test agent reduces or inhibits biofilm formation.
